(19) United States
(12) Patent Application Publication
(10) Pub. No.: US 2006/0167310 A1
(43) Pub. Date: Jul. 27, 2006

(54) Preparation method of naphthalene dicarboxylic acid

(76) Inventors: Jong-In Lee, Gyeonggi-do (KR); Han-Seok Kim, Gyeonggi-do (KR); Byung-Hee Kim, Gyeonggi-do (KR); Hang-Duk Roh, Gyeonggi-do (KR); Youn-Seo Lee, Gyeonggi-do (KR); Joon-Sang Jo, Gyeonggi-do (KR)

Correspondence Address: GIFFORD, KRASS, GROH, SPRINKLE & CITKOWSKI, P.C, PO BOX 7021, TROY, MI, 48007-7021, US

(21) Appl. No.: 10/523,874

(22) PCT Filed: May 2, 2003
(86) PCT No.: PCT/KR03/00883

§ 371 Date: Feb. 7, 2005

(30) Foreign Application Priority Data
Aug. 8, 2002 (KR) 10-2002-0046765

(51) Int. Cl. C07C 51/255

(52) U.S. Cl. 562/416

(57) ABSTRACT

The present invention relates to a method for the preparation of naphthalene dicarboxylic acid, and more particularly, to a method for the preparation of naphthalene dicarboxylic acid by oxidizing dimethylnaphthalene with oxygen in air in the presence of acetic acid solvent using the metal catalysts of cobalt and manganese, and using bromine as a reaction initiator, wherein the temperature of said oxidation reaction is 155 to 180° C. The method for the preparation of naphthalene dicarboxylic acid of the invention enables the preparation of naphthalene dicarboxylic acid having high purity with a high yield in an economical way at a low temperature.

### BACKGROUND OF THE INVENTION

(a) Field of the Invention

The present invention relates to a method for the preparation of naphthalene dicarboxylic acid, and more particularly, to a method that is capable of preparing naphthalene dicarboxylic acid having high purity with a high yield in an economical way at a low temperature.

(b) Description of the Related Art

Naphthalene dicarboxylic acid (NDA) is a raw material of polyethylene naphthalate resin, which is a polyester resin. Of the polyester resins, polyethylene terephthalate resin is now widely used for industrial purposes. This is due to the fact that polyethylene terephthalate is inexpensive and thus enables the preparation of polyester at a low cost even though its properties are poor as compared with polyethylene naphthalate resin.

However, polyethylene naphthalate resin is excellent in several properties such as thermal resistance, tensile strength, impact strength, and gas impermeability in comparison with the polyethylene terephthalate. Such excellent properties of polyethylene naphthalate are known to be due to the rigidity of the double-ring structure contained within the polyethylene naphthalate. One of the monomers that is most widely used for the preparation of such polyethylene naphthalate resin is 2,6-naphthalene dicarboxylic acid.

In general, methods for the preparation of 2,6-naphthalene dicarboxylic acid that have been widely known so far include oxidizing 2,6-dimethyl naphthalene (DMN), 2,6-diisopropyl naphthalene, and 6-methyl-2-acetonaphthalene, etc. as starting materials with oxygen in air.

U.S. Pat. No. 4,950,786 describes that 2,6-naphthalene dicarboxylic acid can be prepared with a high yield by oxidizing 2,6-diisopropyl naphthalene or its oxidative derivatives as starting materials with oxygen in air in the presence of acetic acid solvent, and by further adding cerium or iron, etc. to a cobalt, manganese, and bromine catalyst system in this oxidation. However, the actual preparation of 2,6-naphthalene dicarboxylic acid according to this method revealed that unlike as described in the specification of U.S. Pat. No. 4,950,786, the yield did not exceed 80%, and impurities that deteriorate the activity of the catalyst such as trimellitic acid (TMLA) were generated in large quantities, and thus 2,6-naphthalene dicarboxylic acid could not be produced with a high yield and with high purity.

The method of preparing 2,6-naphthalene dicarboxylic acid using 2,6-diisopropyl naphthalene as a starting material as shown in U.S. Pat. No. 4,950,786 was spotlighted in the past because of the supply and demand problems of raw materials, but as the yield is low and a number of byproducts are generated as described above, the method of preparing 2,6-naphthalene dicarboxylic acid using 2,6-diisopropyl naphthalene as a starting material is hardly used at the present time. Thus, a method of preparing 2,6-naphthalene dicarboxylic acid using 2,6-dimethylnaphthalene as a starting material is now widely used.

U.S. Pat. No. 3,870,754 discloses a method capable of preparing 2,6-naphthalene dicarboxylic acid with a high yield of not less than 90% by oxidizing 2,6-dimethylnaphthalene as a starting material with oxygen in air in the presence of acetic acid solvent and cobalt, manganese, and bromine catalysts. In such method described in U.S. Pat. No. 3,870,754, however, the molar ratio of 2,6-dimethylnaphthalene to acetic acid is restricted so that it cannot exceed 1:100, preferably 1:200. Due to such restriction on the molar ratio of 2,6-dimethylnaphthalene to acetic acid, the production amount of 2,6-naphthalene dicarboxylic acid is substantially reduced, and filtration and solvent treatment processes after oxidation are costly.

Also, U.S. Pat. No. 5,183,933 discloses a method capable of preparing 2,6-naphthalene dicarboxylic acid with a high yield without adding catalysts other than cobalt, manganese, and bromine catalysts having a high concentration of not less than 0.7%, for the oxidation reaction of 2,6-dimethylnaphthalene at a high temperature of not less than 190° C. However, as in the method disclosed in U.S. Pat. No. 5,183,933, the temperature for oxidation is too high and the concentration of the catalysts is too high, so the manufacturing costs are increased and it is thus economically undesirable.

During the oxidation reaction of dimethylnaphthalene, various byproducts and reaction intermediates are generated. For example, trimellitic acid is generated by the oxidation of one of the naphthalene rings, 2-formyl-6-naphthoic acid (FNA) is generated by the incomplete oxidation of dimethylnaphthalene, bromo 2,6-naphthalene dicarboxylic acid (Br-NDA) is generated by the bromination of naphthalene rings, and 2-napthoic acid is generated by the loss of a methyl group or carboxylic acid group-among the substitutes. In addition, unidentified impurities are generated, but the above-exemplified impurities can be said to be typical. The above impurities deteriorate the activity of the catalysts and also deteriorate the purity and yield of the naphthalene dicarboxylic acid to be prepared.

In particular, of the impurities, the trimellitic acid forms a complex compound along with the metal catalysts of cobalt and manganese used in the oxidation, thereby deteriorating the activity of these catalysts.

Further, as the naphthalene dicarboxylic acid prepared by the oxidation of dimethylnaphthalene shows very low solubility in water, acetic acid, and aliphatic or aromatic hydrocarbons, etc., the elimination of impurities is not easy in purification processes such as recrystallization or adsorption, and thus the purity of the naphthalene dicarboxylic acid to be prepared is low.

### SUMMARY OF THE INVENTION

This invention has been made to solve the problems as described above, and it is an object of the invention to provide a method for the preparation of naphthalene dicarboxylic acid having high purity with a high yield in an economical way at a low temperature.

In order to achieve the aforementioned object, the present invention provides a method for the preparation of naphthalene dicarboxylic acid by oxidizing dimethylnaphthalene with oxygen in air in the presence of acetic acid solvent using the metal catalysts of cobalt and manganese, and using bromine as a reaction initiator, wherein the temperature of said oxidation reaction is 155 to 180° C.

### BRIEF DESCRIPTION OF THE DRAWINGS

FIG. 1 shows a semi-continuous-type reactor for the preparation of naphthalene dicarboxylic acid.

FIG. 2 shows a continuous-type reactor for the preparation of naphthalene dicarboxylic acid.

### DETAILED DESCRIPTION OF THE PREFERRED EMBODIMENTS

The present invention will be hereafter described in more detail.

The method for the preparation of naphthalene dicarboxylic acid of the invention is conducted by the liquid-state oxidation reaction of dimethylnaphthalene with oxygen in air in the presence of acetic acid solvent using metal catalysts of cobalt and manganese, and using bromine as a reaction initiator, and it is characterized in that the temperature of said oxidation reaction is 155 to 180° C.

The prepared naphthalene dicarboxylic acid is more preferably 2,6-naphthalene dicarboxylic acid.

As criteria capable of assessing the quality of the naphthalene dicarboxylic acid to be prepared, concentrations of trimellitic acid and 2-formyl-6-naphthoic acid, which are impurities generated during the oxidation reaction, and color-b that indicates the content of bromine, etc., are evaluated. High color-b of the products indicates that impurities comprising bromine compounds are contained in large quantities within the products, and it makes the products dark brown, which is not desirable.

The method for the preparation of naphthalene dicarboxylic acid of the invention is characterized in that the temperature of the oxidation reaction is 155 to 180° C. If the temperature of the oxidation reaction is less than 155° C., the content of the impurity 2-formyl-6-naphthoic is excessively increased and the purity of the naphthalene dicarboxylic acid to be produced is thus deteriorated, and if the temperature of the oxidation reaction exceeds 180° C., the content of the impurity trimellitic acid is excessively increased and the impurity bromine compound is contained in large quantities within the products, and thus the purity of the naphthalene dicarboxylic acid to be produced is deteriorated.

Further, in the method for the preparation of naphthalene dicarboxylic acid of the invention, the concentration of the metal catalysts of cobalt and manganese is 1000 ppm to 6000 ppm in the acetic acid solvent.

If the concentration of the metal catalysts of cobalt and manganese is less than 1000 ppm in the acetic acid solvent, the oxidation reaction of the starting material dimethylnaphthalene, does not proceed smoothly, and if the concentration of the metal catalysts of cobalt and manganese exceeds 6000 ppm in the acetic acid solvent, the yield of naphthalene dicarboxylic acid is not increased simply in proportion to the catalysts to be fed, and thus it is not desirable in economical aspects.

Also, the method for the preparation of naphthalene dicarboxylic acid of the invention is characterized in that the molar ratio of the metal catalysts of cobalt to manganese is 2:1 to 25:1. If the molar ratio of cobalt with regard to manganese is less than 2, the oxidation reaction of dimethylnaphthalene does not proceed smoothly, and if the molar ratio of cobalt with regard to manganese exceeds 25, the loss of acetic acid is excessively increased even though the oxidation reaction of dimethylnaphthalene proceeds smoothly, and accordingly, it is not desirable.

Also, the method for the preparation of naphthalene dicarboxylic acid of the invention is characterized in that the molar ratio of the bromine to the metal catalysts of cobalt and manganese is 0.1:1 to 0.8:1. If the molar ratio of bromine with regard to the metal catalysts of cobalt and manganese is less than 0.1, the production of 2-formyl-6-naphthoic acid, which is a reaction intermediate and major impurity, is excessively increased, and thus it is not advisable because the purity and yield of the naphthalene dicarboxylic acid to be produced are deteriorated, and if the molar ratio of bromine with regard to the metal catalysts of cobalt and manganese exceeds 0.8, the bromine compound impurity is excessively generated in large quantities within the naphthalene dicarboxylic acid to be produced, and thus it is not advisable because the purity and yield of naphthalene dicarboxylic acid are deteriorated.

Also, the method for the preparation of naphthalene dicarboxylic acid of the invention is characterized in that the residence time of said acetic acid solvent and the produced naphthalene dicarboxylic acid in the reactor is 30 min. to 120 min.

If the residence time of the acetic acid solvent and the produced naphthalene dicarboxylic acid in the reactor is less than 30 min., unreacted reaction intermediates are increased and thus it is not desirable, and if the residence time of the acetic acid solvent and the produced naphthalene dicarboxylic acid in the reactor exceeds 120 min., the consumption amount of the acetic acid solvent is increased because of the increase of its oxidation reaction and thus it is not desirable.

Also, the method for the preparation of naphthalene dicarboxylic acid of the invention is characterized in that said air to dimethylnaphthalene ratio is 4:1 to 15:1 by weight, and nitrogen, an off-gas where the concentration of oxygen is lowered after oxidation reaction, or a mixture thereof is charged into the upper portion of the reactor. If the weight ratio of air with regard to the starting material dimethylnaphthalene is less than 4, the amount of oxygen is not sufficient and thus the oxidation reaction of dimethylnaphthalene does not proceed smoothly, and if the weight ratio of air with regard to dimethylnaphthalene exceeds 15, the oxidation reaction rate of dimethylnaphthalene is not increased simply in proportion to the weight ratio of oxygen, and excessive investment costs are required for the facilities such as an air compressor for supplying a large quantity of compressed air, a reactor, a heat exchanger, etc., and thus it is not advisable in economical aspects.

Also, the charge of nitrogen, an off-gas where the concentration of oxygen is lowered after oxidation reaction, or a mixture thereof into the upper portion of the reactor can prevent a risk of explosion inside the reactor owing to excess oxygen.

As described in the above, the method for the preparation of naphthalene dicarboxylic acid of the invention enables the preparation of naphthalene dicarboxylic acid having high purity with a high yield in an economical way at a low temperature.

The following are preferred examples of the invention and comparative examples. The following examples and comparative examples are provided solely to describe the invention more clearly; the matter of the invention should not be construed to be limited thereto.

The metal catalysts in the following examples and comparative examples refer to cobalt and manganese, and their concentration is based on the weight of a solvent and refers to a weight concentration occupied by each atom. The cobalt and manganese used in oxidation reaction experiments may be used in the form of various compounds, but in the following examples and comparative examples, cobalt, manganese, and bromine were used in the form of cobalt hydroxide (Co(OH)₂), manganese acetate tetrahydrate (Mn(OAc)₂4H₂O), and hydro bromic acid (HBr 48% solution), respectively.

Also, the ratio of the metal catalysts is the molar concentration of cobalt divided by the molar concentration of manganese, and it indicates a molar ratio of cobalt to manganese contained in the solvent. Likewise, the concentration of bromine used as a reaction initiator is based on the solvent, and it refers to a weight concentration of bromine atoms contained in the solvent, while the ratio of bromine/metal catalysts is the molar concentration of bromine contained in the solvent divided by the molar concentration of the metal catalysts (cobalt and manganese).

The slurries generated during the oxidation reaction according to the conditions of the following examples and comparative examples were separated into solid/liquid, and then a portion of the solid was subjected to an organic-impurities analysis using gas chromatography after a drying procedure, and the remaining solid was subjected to color determination after washing, separation into solid/liquid, and drying procedures.

Concentrations of trimellitic acid, 2-formyl-6-naphthoic acid, etc., which are major impurities within naphthalene dicarboxylic acid, were determined using gas chromatography. Of the color determination results, color-b is an index indicating the yellowness of products, and the higher the value is, the thicker yellow it indicates.

Naphthalene dicarboxylic acid having yellow impurities has an effect on the polymerization product to be produced therefrom, and thus it generates undesirable polyethylene naphthalate whose color-b shows high yellow, which is not desirable. In this respect, so as to be used as raw materials for polymerization, a method is required for the preparation of naphthalene dicarboxylic acid having a good quality whose color-b value is low, as shown in the present invention.

### EXAMPLES 1 TO 2 AND COMPARATIVE EXAMPLES 1 TO 2

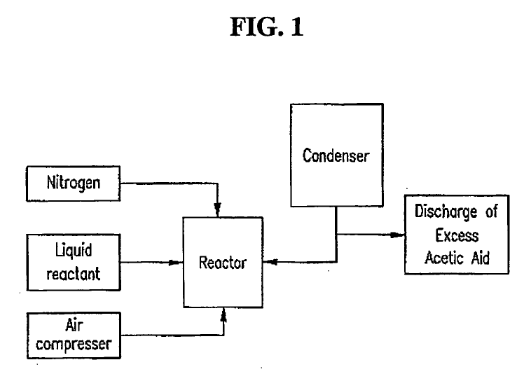

Semi-continuous oxidation reaction experiments were carried out in a semi-continuous-type reactor for the preparation of naphthalene dicarboxylic acid, as shown in FIG. 1. First, the metal catalysts (cobalt and manganese) were dissolved in 1 kg of acetic acid in such a suitable amount that their concentration became 3000 ppm on the basis of atoms, and then injected into a 2-liter titanium reactor.

Nitrogen was charged into the titanium reactor until the pressure within the reactor became 6 atmospheres and then the temperature of the reactor was raised up to 160 C. The reactor was installed with a heating jacket and a cooling coil, and the reaction temperature was maintained within the range of ±2° C. by a thermostat. Also, the reaction pressure was constantly maintained using a back pressure regulator that was installed in the rear part of a condenser.

When the reactor reached the reaction temperature and pressure, 50 g of dimethylnaphthalene was dissolved in 1 kg of acetic acid, which was then injected using a quantitative pump at a speed of 20 g/min into the reactor, and at the same time, air, which was compressed in an air compressor, was fed at a constant amount into the reactor, whereby an oxidation reaction was carried out.

During the oxidation reaction, so as to constantly maintain the concentration of the catalyst and the water level within the reactor, excess acetic acid was discharged to the outside of the system from the lower portion of the condenser. After the liquid reactants were all fed into the reactor, in order to terminate the reaction, air was additionally injected for 30 min. while maintaining the reaction temperature and pressure.

To examine an effect of the concentration of bromine on the oxidation reaction of 2,6-dimethylnaphthalene, the oxidation reaction was carried out while varying the concentration of bromine to the concentration of the metal catalysts, and the quality of the obtained 2,6-naphthalene dicarboxylic acid was determined. The results are shown in Table 1 below. In addition, the following Table 1 shows the reaction conditions of Examples 1 and 2 and Comparative Examples 1 and 2.

TABLE 1

| | | | | Compara- | Compara- |
|---|---|---|---|---|---|
| | | Ex. 1 | Ex. 2 | tive Ex. 1 | tive Ex. 2 |
| Reaction | Bromine/Metal | 0.3 | 0.8 | 0.06 | 1 |
| Condi- | Catalyst | | | | |
| tions | Cobalt/ | 20 | 20 | 20 | 20 |
| | Manganese | | | | |
| Reaction | 2-Formyl-6- | 1805 | 1330 | 9058 | 1742 |
| Results | naphthoic acid | | | | |
| | (ppm) | | | | |
| | Trimellitic acid | 3199 | 2611 | 4354 | 3327 |
| | (ppm) | | | | |
| | Color-b | 9.33 | 12.04 | 10.94 | 17.74 |

As shown in Comparative Example 1 of Table 1 above, when the concentration of bromine was too low, the concentration of 2-formyl-6-naphthoic acid, which is the reaction intermediate and also a major impurity, was high. Considering that the purification of naphthalene dicarboxylic acid is difficult in a purification process after the oxidation process, because naphthalene dicarboxylic acid has a low solubility in acetic acid which is a reaction medium, we can conclude that a high concentration of 2-formyl-6-naphthoic acid, which is an impurity, is not desirable.

Also, as shown in Comparative Example 2 of Table 1 above, when the concentration of bromine was too high, color-b of the produced naphthalene dicarboxylic acid was too high and it appeared as a dark brown powder, even visually. Therefore, we can conclude that in such a case, naphthalene dicarboxylic acid having good quality cannot be produced.

### EXAMPLES 3 TO 5 AND COMPARATIVE EXAMPLE 3

The procedures were carried out in the same manner as in Example 1 above, except that the ratio of solvents varied by each example and comparative example, and the conditions and results are shown in Table 2 below. The ratio of solvents, as described above, is the weight of acetic acid contained in the liquid mixture to be fed divided by the weight of a reactant, 2,6-dimethylnaphthalene.

TABLE 2

| | | | | | Compara- |
|---|---|---|---|---|---|
| | | Ex. 3 | Ex. 4 | Ex. 5 | tive Ex. 3 |
| Reaction | Ratio of Solvents | 20 | 10 | 50 | 3 |
| Condi- | Bromine/Metal | 0.8 | 0.7 | 0.7 | 0.7 |
| tions | Catalyst | | | | |
| | Cobalt/Manganese | 20 | 20 | 20 | 20 |
| Reaction | 2-Formyl-6- | 1330 | 4173 | 1735 | 12870 |
| Results | naphthoic acid | | | | |
| | (ppm) | | | | |
| | Trimellitic acid | 2611 | 3251 | 1657 | 5537 |
| | (ppm) | | | | |
| | Color-b | 11.57 | 12.04 | 10.08 | 22.34 |

As shown in Comparative Example 3 of Table 2 above, when the ratio of solvents was 3, color-b was 22.34 and the product was dark brown. It can be said that the higher the ratio of solvents is, the higher quality can be obtained in every aspect including organic impurities, color, etc. However, as the ratio of solvents is increased, manufacturing costs are increased, and accordingly, a high ratio of solvents is not necessarily advisable.

### EXAMPLES 6 TO 8 AND COMPARATIVE EXAMPLE 4

The procedures were carried out in the same manner as in Example 1 above, except that the ratio of metal catalysts varied by each example and comparative example, and the results are shown in Table 3 below.

TABLE 3

| | | | | | Compara- |
|---|---|---|---|---|---|
| | | Ex. 6 | Ex. 7 | Ex. 8 | tive Ex. 4 |
| Reaction | Bromine/Metal | 0.7 | 0.7 | 0.7 | 0.7 |
| Condi- | Catalyst | | | | |
| tions | Cobalt/Manganese | 2 | 4 | 20 | 1 |
| Reaction | 2-Formyl-6- | 4085 | 2913 | 2466 | 8120 |
| Results | naphthoic acid | | | | |
| | (ppm) | | | | |
| | Trimellitic acid | 5080 | 3430 | 3374 | 9763 |
| | (ppm) | | | | |
| | Color-b | 16.05 | 12.41 | 11.84 | 21.58 |

As shown in Table 3 above, as the ratio of cobalt/manganese was increased, the oxidation reaction rate was increased. When cobalt and manganese were injected at the same molar concentration, as shown in Comparative Example 4, the production amount of the reaction intermediates, 2-formyl-6-naphthoic acid and trimellitic acid, was increased. Also, the color-b was as high as 21.58.

### EXAMPLES 9 AND 10 AND COMPARATIVE EXAMPLES 5 AND 6

The procedures were carried out in the same manner as in Example 1 above, except that the reaction temperature varied by each example and comparative example, and the results are shown in Table 4 below.

TABLE 4

| | | | | Compara- | Compara- |
|---|---|---|---|---|---|
| | | Ex. 9 | Ex. 10 | tive Ex. 5 | tive Ex. 6 |
| Reaction | Reaction | 160 | 180 | 140 | 190 |
| Condi- | Temperature | | | | |
| tions | (° C.) | | | | |
| | Bromine/Metal | 0.7 | 0.7 | 0.7 | 0.7 |
| | Catalyst | | | | |
| | Cobalt/ | 20 | 20 | 20 | 20 |
| | Manganese | | | | |
| Reaction | 2-Formyl-6- | 2466 | 1529 | 15361 | 1311 |
| Results | naphthoic acid | | | | |
| | (ppm) | | | | |
| | Trimellitic acid | 3374 | 5379 | 3150 | 12326 |
| | (ppm) | | | | |
| | Color-b | 11.84 | 17.50 | 10.89 | 23.70 |

As shown in Comparative Example 5 in Table 4 above, when the reaction temperature was less than 155° C., the production amount of a reaction intermediate, 2-formyl-6-naphthoic acid was excessively high, and as shown in Comparative Example 6, when the reaction temperature exceeds 180° C., the production amount of a reaction intermediate, trimellitic acid was excessively high, and accordingly, we can conclude that neither case is desirable.

### EXAMPLES 11 AND 12 AND COMPARATIVE EXAMPLE 7

The procedures were carried out in the same manner as in Example 1 above, except that the concentration of metal catalysts varied by each example and comparative example, and the results are shown in Table 5 below.

TABLE 5

| | | | | Comparative |
|---|---|---|---|---|
| | | Ex. 11 | Ex. 12 | Ex. 7 |
| Reaction | Concentration of Catalyst | 3000 | 6000 | 10000 |
| Conditions | (ppm) | | | |
| | Bromine/Metal Catalyst | 0.3 | 0.3 | 0.3 |
| | Cobalt/Manganese | 20 | 20 | 20 |
| Reaction | 2-Formyl-6-naphthoic acid | | | |
| Results | (ppm) | 1805 | 2008 | 1725 |
| | Trimellitic acid (ppm) | 3199 | 2326 | 5082 |
| | Color-b | 9.33 | 13.74 | 22.38 |

As shown in Comparative Example 7 of Table 5 above, when the concentration of the total catalysts was increased, the concentration of 2-formyl-6-naphthoic acid had a tendency of being slightly decreased, but the value of color-b and trimellitic acid was excessively high. This shows that if the concentration of the total catalyst becomes high, the absolute amount of the total bromine to be injected so as to meet the ratio of bromine is increased, and as a result, the concentration of bromine compounds in the produced naphthalene dicarboxylic acid is also substantially increased.

### EXAMPLES 13 TO 17

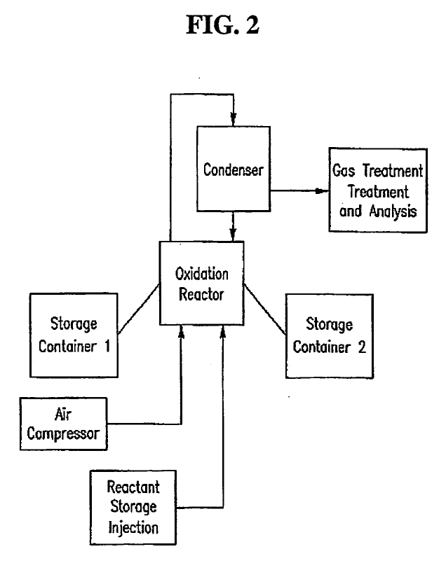

FIG. 2 shows a continuous-type reactor for the preparation of naphthalene dicarboxylic acid. The mixed liquid reactants consisting of acetic acid, 2,6-dimethylnaphthalene, catalysts, etc. were fed into the oxidation reactor constantly at a desired flow rate using a quantitative pump.

The gaseous phase reactant, air, was compressed to a desired pressure using a compressor and then injected into the oxidation reactor at a constant flow rate via a flow rate control valve.

The oxidation reactor was heated up to the reaction temperature using the heating jacket, and the reaction temperature and pressure were controlled by a pressure control valve that was installed in the rear part of the condenser. The 2,6-naphthalene dicarboxylic acid/acetic acid slurry that was produced under such conditions was sent to storage container 1 until the system reached its normal state, and after reaching its normal state, the slurry was sent to storage container 2, where a sample thereof was collected.

After the collected 2,6-naphthalene dicarboxylic acid slurry was subjected to filtration, washing, and drying processes, the content of organic impurities and color-b contained therein were assayed.

In addition, excess gas and vapor, which were generated in the oxidation reactor or left after participation in the reaction, were passed through the condenser where the vapor phase was condensed and returned, but most gas whose temperature dropped in the condenser was discharged to atmosphere, and some gas was subjected to a process for eliminating the remaining vapor and then assayed on-line.

The concentrations of oxygen, carbon dioxide, and carbon monoxide contained in the discharge gas were determined in the state that vapor was completely eliminated. Carbon dioxide and carbon monoxide are generated in the oxidation process of acetic acid that is used as a reaction medium, and they have an adverse effect on the manufacturing process of 2,6-naphthalene dicarboxylic acid by inducing the loss of acetic acid.

Table 6 below shows oxidation reaction conditions and results in each example.

TABLE 6

| | | Ex. 13 | Ex. 14 | Ex. 15 | Ex. 16 | Ex. 17 |
|---|---|---|---|---|---|---|
| Reaction | Ratio of | 25 | 10 | 5 | 10 | 10 |
| Condi- | Solvents | | | | | |
| tions | (by weight) | | | | | |
| | Residence | 90 | 90 | 90 | 60 | 90 |
| | Time (min.) | | | | | |
| | Air/ | 5 | 5 | 5 | 5 | 15 |
| | Dimethyl- | | | | | |
| | naphthalene | | | | | |
| | (by weight) | | | | | |
| Reaction | 2-Formayl-6- | 3695 | 4282 | 7835 | 4282 | 3312 |
| Results | naphthoic | | | | | |
| | acid (ppm) | | | | | |
| | Color-b | 10.85 | 12.92 | 15.38 | 14.78 | 10.40 |

As shown in Example 15 of Table 6 above, when the ratio of solvents was 5, an intermediate product, 2-formyl-6-naphthoic acid and color-b were increased, but nevertheless, 2,6-naphthalene dicarboxylic acid having a high quality in comparison with Comparative Example 3 was obtained.

Also, as shown in Example 16, when the residence time was reduced to 60 min., comparatively good results were obtained in view of the concentration of 2-formyl-6-naphthoic acid in spite of the increase of color-b, and as shown in Example 17, when the ratio of air/dimethylnaphthalene by weight was increased, the concentration of the intermediate, 2-formyl-6-naphthoic acid, and color-b value were all decreased.

However, since there is a risk of explosion if excess oxygen is fed into the reactor, it is advisable to charge pure nitrogen into the upper portion of the reactor or re-charge off-gas where the concentration of oxygen is lowered by reaction into the upper portion of the reactor.

As described in the above, the method for the preparation of naphthalene dicarboxylic acid of the invention enables the preparation of naphthalene dicarboxylic acid having high purity with high yield In an economical way at a low temperature.

1. A method for the preparation of naphthalene dicarboxylic acid comprising: oxidizing dimethylnaphthalene with oxygen in air in a reactor and in the presence of acetic acid solvent, metal catalysts of cobalt and manganese, and bromine as,
wherein the oxidizing reaction is carried out in the range of 155 to 180° C. and in the presence of a gas selected from the group of nitrogen gas, off-gas and a mixture thereof into an upper portion of said reactor, to yield a naphthalene dicarboxylic acid.

2. The method for the preparation of naphthalene dicarboxylic acid of claim 1, wherein said naphthalene dicarboxylic acid is 2,6-naphthalene dicarboxylic acid.

3. The method for the preparation of naphthalene dicarboxylic acid of claim 1, wherein the concentration of said metal catalysts of cobalt and manganese is 1000 ppm to 6000 ppm in acetic acid.

4. The method for the preparation of naphthalene dicarboxylic acid of claim 1, wherein the molar ratio of said metal catalysts of cobalt and manganese is 2:1 to 25:1.

5. The method for the preparation of naphthalene dicarboxylic acid of claim 1, wherein the molar ratio of said bromine to the metal catalysts of cobalt and manganese is 0.1:1 to 0.8:1.

6. The method for the preparation of naphthalene dicarboxylic acid of claim 1, wherein the residence time of said acetic acid and said naphthalene dicarboxylic acid in the reactor is 30 to 120 min.

7. The method for the preparation of naphthalene dicarboxylic acid of claim 1, wherein the weight ratio of said air to dimethylnaphthalene is 4:1 to 15:1.

8. The method for the preparation of naphthalene dicarboxylic acid of claim 1, wherein the off-gas has an oxygen concentration lowered by pre-oxidation in the upper portion of said reactor.
